(19) United States
(12) Patent Application Publication
(10) Pub. No.: US 2006/0183723 A1
(43) Pub. Date: Aug. 17, 2006

(54) Ethercarboxylic acid ester of sterol or stanol

(76) Inventors: Alfred Westfechtel, Hilden (DE); Ansgar Behler, Bottrop (DE)

Correspondence Address: COGNIS CORPORATION;PATENT DEPARTMENT, 300 BROOKSIDE AVENUE, AMBLER, PA, 19002, US

(21) Appl. No.: 10/549,744

(22) PCT Filed: Mar. 10, 2004
(86) PCT No.: PCT/EP04/02440

§ 371 Date: Sep. 19, 2005

(30) Foreign Application Priority Data
Mar. 19, 2003 (DE) 103 12 000.9

(51) Int. Cl. A61K 31/56; C07J 9/00

(52) U.S. Cl. 514/169; 552/540

(57) ABSTRACT

The disclosed invention relates to sterol and/or stanol esters of ethercarboxylic acids corresponding to general formula (I):R(OAlk)ₙOCH₂COOH, in which R is a C₁₋₅₀ alkyl, alkenyl and/or alkylphenyl group derived from an alcohol, OAlk stands for ring-opened ethylene oxide, propylene oxide and/or butylene oxide units and n is a number of 0 to 100, and to a process for making said sterols and stanols. The inventive sterols and stanols have a number of uses, including as steroid precursors, as emulsifiers and as a hypocholesterolaemic active component of foods.

### FIELD OF THE INVENTION

This invention relates generally to esters of sterols and/or stanols and, more particularly, to new ethercarboxylic acids esters, to a process for their production and to the use of these esters.

### PRIOR ART

In recent years, sterols and particularly phytosterols have received increasing attention. Thus, they are used to a large extent in fermentation to steroid intermediates, such as androstendione, which in turn is converted by mostly chemical modifications into pharmaceutically active steroids, such as testosterone, estradiol, testolactone or cortisone. In addition, phytosterols have the advantageous property of lowering the cholesterol level in human blood, so that they—and their fatty acid esters—are used as a food additive.

One of the major problems of using sterols and the stanols obtainable by hydrogenation has been their extremely poor solubility in water and their minimal solubility in fats. One way of producing aqueous dispersions is reducing the particle size of the sterols using various high-pressure homogenizers. Thus, the production of microparticles of phytosterols inter alia in an aqueous medium by using high-shear apparatus, such as colloid mills, is known from International patent application WO 00/45648.

Another method of producing aqueous dispersions of sterols is to use emulsifiers, generally followed by homogenization, for example in accordance with European patent EP-B-897 671.

European patent application EP-A-195311 describes sterol derivatives with improved solubility in fats which can be produced by esterification of phytosterols and branched aliphatic alcohols with fatty acids or fatty acid esters in the presence of lipases.

German patent application DE-A-2035069 discloses clear cooking oils and salad dressing containing carboxylic acid esters of phytosterols produced by acylation using perchloric acid as catalyst.

In addition, β-sitostanol fatty acid esters produced by transesterification of β-sitostanol with fatty acid esters in the presence of transesterification catalysts are known from International patent application WO 92/19640.

The problem addressed by the present invention was to provide sterol derivatives and/or stanol derivatives which would be dispersible in large quantities in water without any need for expensive high-pressure homogenizers in order to open up a broad range of applications. At the same time, the derivatives would be self-dispersible, i.e. the presence of emulsifiers for the production of aqueous dispersions would be merely optional rather than compulsory. In addition, the derivatives would have a lower melting point than the sterols and/or stanols themselves, for example to simplify fermentative consequent reactions. Finally, the invention sought to provide derivatives which would be capable as required of releasing the sterols and/or stanols again in aqueous media under controlled conditions.

### DESCRIPTION OF THE INVENTION

The present invention relates to sterol and/or stanol esters of ethercarboxylic acids corresponding to general formula (I):
R(OAlk)ₙOCH₂COOH (I)
in which R is a C₁₋₅₀ alkyl, alkenyl and/or alkylphenyl group derived from an alcohol, OAlk stands for ring-opened ethylene oxide, propylene oxide and/or butylene oxide units and n is a number of 0 to 100.

The present invention also relates to a process for the production of sterol and/or stanol esters of ethercarboxylic acids which is characterized in that sterols and/or stanols are esterified with ethercarboxylic acids or their salts corresponding to formula (II):
R(OAlk)ₙOCH₂COOX (II)
in which R is a C₁₋₅₀ alkyl, alkenyl and/or alkylphenyl group derived from an alcohol, OAlk stands for ring-opened ethylene oxide, propylene oxide and/or butylene oxide units, n is a number of 0 to 100 and X is hydrogen or an alkali metal,
in the presence of an esterification catalyst.

The present invention also relates to the use of sterol and/or stanol esters of ethercarboxylic acids corresponding to formula (I) as a sterol and/or stanol source, preferably as a raw material for the production of steroid precursors, more particularly for the fermentative production of 4-androsten-3,17-dione (AD) and/or 4-androstadien-3,17-dione (ADD), as emulsifiers, more particularly in cosmetic preparations and in foods, as a cosmetic active component and, finally, as a hypocholesterolaemic active component, more particularly in foods and/or food supplements.

Sterols and Stanols

Sterols, which are also often referred to as sterins, are C₂₇₋₃₀ steroids with a hydroxyl group at the third carbon atoms. In general, sterols also have a double bond. Stanols are the corresponding saturated sterol compounds which do not have a double bond. The sterols occur widely in nature as esters or glycosides. There are animal sterols, so-called zoosterols, vegetable sterols, so-called phytosterols, and sterols from fungi, so-called mycosterols. In principle, any sterols or stanols, but preferably the phytosterols or their hydrogenated stanol compounds, are suitable for the purposes of the invention. Phytosterols which mainly contain one or more of the following compounds: β-sitosterol, campesterol, stigmasterol, brassicasterol, stigmasterol and campestanol, are preferred.

Mixtures of sterols and/or stanols which contain at least two of the following compounds are particularly suitable for the purposes of the invention: β-sitosterol, campesterol, β-sitostanol and/or campestanol which have the following formulae:

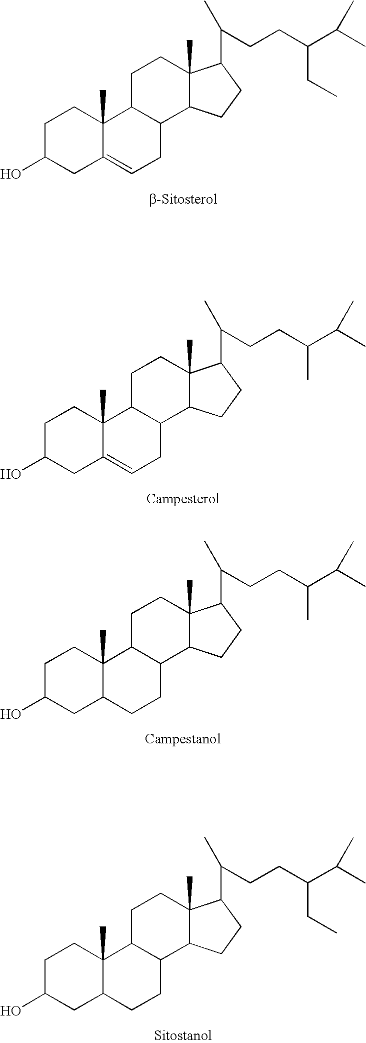

Phystosterols derivatized from vegetable oils, more particularly from rapeseed oil, are most particularly preferred for the purposes of the invention. The sterol mixtures marketed by Cognis Corporation under the names of Generol™100 and Generol™122 are particularly suitable. Generol™100 is a mixture which is rich in beta-sitosterol, campesterol and stigmasterol (ca. 80% by weight) and which additionally contains brassicasterol (ca. 5% by weight), stigmasterol (ca. 12% by weight) and campestanol (balance to 100% by weight). Generol™122 is also a mixture which contains 40 to 55% by weight beta-sitosterol, 20 to 28% by weight campesterol, 14 to 23% by weight stigmasterol and, optionally, 0 to 8% by weight brassicasterol, 0 to 5% by weight stigmasterol and 0 to 2% by weight campestanol. In addition, Generol RTM, a mixture of 40 to 60% by weight beta-sitosterol, 30 to 45% by weight campesterol and 8 to 18% by weight brassicasterol, 0 to 5% by weight stigmasterol, 0 to 5% by weight stigmastanol, 0 to 5% by weight ergostanol and a total content of 90 to 100% by weight which is marketed by Cognis Deutschland GmbH & Co. KG is also particularly suitable.

Ethercarboxylic Acids

According to the invention, the sterols and/or stanols are esterified with ethercarboxylic acids corresponding to formula (I), i.e. the hydroxyl group of the sterols and/or stanols forms an ester group with the ethercarboxylic acid.

The ethercarboxylic acids, which are known per se, are produced from alcohols which are optionally alkoxylated in the first process step and then carboxymethylated with a halocarboxylic acid or salts thereof. This process is described, for example, in German patent DE-C-197 40 954.

The alcohols on which the later ethercarboxylic acid is based may be aliphatic, cycloaliphatic or even aromatic and may be both saturated and unsaturated. Linear aliphatic C₁₋₃₆ alcohols optionally containing another functional group (i.e. R in formula (I) is a linear C₁₋₃₆ alkyl group derived from an alcohol and optionally containing other functional groups) have been found to be suitable. The other functional groups are preferably hydroxyl groups, more particularly terminal hydroxyl groups. In other words, the alcohols on which the ethercarboxylic acids are based may be diols, i.e. difunctional alcohols preferably containing 2 to 36 carbon atoms. In this case, R in general formula (I) is a linear, hydroxyl-terminated C₂₋₃₆ alkyl group derived from a difunctional alcohol. Examples of such difunctional alcohols are ethylene glycol, propylene glycol, pentane-1,5-diol, octane-1,8-diol, hexane-1,6-diol, decane-1,10-diol, dodecane-1,12-diol or the dimer and/or trimer alcohols known to the expert. Dimer diols/trimer triols are technical mixtures which are obtained by oligomerization of unsaturated C₁₂₋₂₂, preferably C₁₆₋₁₈ fatty acids or methyl esters thereof and subsequent high-pressure hydrogenation.

According to the invention, the ethercarboxylic acids corresponding to general formula (I) are preferably derived from monofunctional aliphatic alcohols. Examples of suitable primary monofunctional linear alcohols are hexanol, heptanol, octanol, nonanol, decanol, undecanol, dodecanol, tridecanol, tetradecanol, pentadecanol, hexadecanol, heptadecanol, octadecanol, nonadecanol, eicosanol, docosanol, tetracosanol, 10-undecen-1-ol, oleyl alcohol, elaidyl alcohol, ricinolyl alcohol, linoleyl alcohol, linolenyl alcohol, gadoleyl alcohol, arachidonyl alcohol, erucyl alcohol, brassidyl alcohol. Examples of suitable primary monofunctional branched alcohols are isononyl alcohol, isotridecyl alcohol or Guerbet alcohols which are obtainable by dimerization of fatty alcohols and which, structurally, are distinguished by the fact that they have a relatively long alkyl group preferably containing 2 to 12 carbon atoms in the a-position to the terminal CH₂OH group. Suitable Guerbet alcohols are 2-hexyldecanol, 2-octyl decanol and 2-hexyldecyl palmitate/stearate, 2-ethylhexanol and propyl heptanol.

Ethercarboxylic acids derived from monofunctional saturated linear C₁₋₁₈ alcohols, i.e. R in general formula (I) is a linear C₁₋₁₈ alkyl group derived from a monofunctional alcohol, are preferred for the purposes of the invention. Particularly suitable ethercarboxylic acids are derived from methanol, ethanol, propanol, butanol, caproic alcohol, caprylic alcohol, capric alcohol, lauryl alcohol, myristyl alcohol, cetyl alcohol and/or stearyl alcohol and the technical mixtures thereof formed, for example, in the high-pressure hydrogenation of technical methyl esters based on fats and oils. Ethercarboxylic acids of linear C₁₋₆ alcohols are particularly preferred.

The ethercarboxylic acids corresponding to general formula (I) contain optionally ring-opened ethylene oxide, propylene oxide and/or butylene oxide units (OAlk); OAlk preferably stands for the ethylene oxide unit OCH₂CH₂. The degree of alkoxylation of the ethercarboxylic acids corresponding to general formula (I) is represented by “n” which is, of course, a pure statistical measure. n is preferably a number of 1 to 20 and, more particularly, 3 to 15.

Particularly suitable ethercarboxylic acids are commercially obtainable, for example, from Kao under the name of Akypo™, such as Akypo LF 1™ (octanol-based ethercarboxylic acid ethoxylated with 5 mol ethylene oxide), Akypo LF 2™ (octanol-based ether carboxylic acid ethoxylated with 8 mol ethylene oxide), Akypo RLM 100™ (C_{12/14}-alcohol-based ethercarboxylic acid ethoxylated with 10 mol ethylene oxide).

Process for the Production of Sterol and/or Stanol Esters of Ethercarboxylic Acids

The sterol and/or stanol esters according to the invention are prepared by esterification of the sterols and/or stanols with ethercarboxylic acids corresponding to formula (II) or salts thereof in the presence of an esterification catalyst.

In general, the sterols and/or stanols and ethercarboxylic acids are used in molar ratios of 2:1 to 1:2, preferably 1.5:1 to 1:1.5 and more particularly 1.3:1 to 1:1.3. In order to obtain complete esterification, it is recommended to use the ethercarboxylic acid in a slight excess. The esterification is carried out in the presence of known esterification catalysts, such as zinc oxalate, zinc oxide or calcium oxide. Reaction temperatures above the melting point and below the decomposition point of the sterols and/or stanols, preferably between 190 and 230° C, are recommended. With an excess of the alcohol component, the degree of esterification can be worked out from the acid value according to DIN 53402. The esterification may be regarded as virtually complete at a residual acid value (AV) below 10 and preferably below 5. With an excess of the acid component, an analogous procedure may be adopted.

In general, the reaction time is about 4 to 6 hours, the quantity of catalyst used, the reaction temperature and the residual hydroxyl value being co-determining factors. If desired, the esterification may be carried out in vacuo. In addition, it can be of advantage to dry the starting materials before the actual esterification.

Commercial Applications

The ethercarboxylic acid esters of the sterols and/or stanols according to the invention are liquid or wax-like products that are easy to melt. The compounds according to the invention are also easy to emulsify in water in the absence of emulsifiers, i.e. they are self-emulsifiable in water. By virtue of these advantageous properties in relation to sterols and/or stanols, the ethercarboxylic acid esters of the sterols and/or stanols according to the invention are a source of sterols and/or stanols, i.e. they may be used for the same applications as the sterols and/or stanols. According to the invention, the ethercarboxylic acid esters of the sterols and/or stanols are preferably used on the one hand as raw material in the fermentative production of 4-androsten-3,17-dione (AD) and/or 4-androstadien-3,17-dione (ADD) which in turn may be used for the production of various steroid derivatives, such as testosterone, estradiol, ethinylestradiol, testolactone, progesterone, cortisone, cortisol, prednisone or even prednisolone. In the fermentative production of AD and ADD, the ethercarboxylic acids of the sterols and/or stanols are reacted and worked up analogously to the conditions disclosed in published German patent application DE 3147834-A which describes the fermentation of cholesterol in the presence of ethoxylated sterols. Fuller information is available to the expert from the article by P. Fernandez et al. “Microbial conversion of steroid compounds: recent developments” in Enzyme and Microbial Techn. 32 (2003), 688-705. If the substances mentioned are to be used as a sterol source, the presence of lipases for ester cleavage is important.

By virtue of their self-emulsifying properties, the ethercarboxylic acid esters of the sterols and/or stanols may be used as emulsifiers, particularly in cosmetic compositions and in foods. They are suitable as emulsifiers for o/w and for w/o emulsions. The quantity used in determined by the particular application and is generally from 0.1 to 10% by weight and preferably from 0.5 to 5% by weight, based on the particular composition. Their use in combination with other, known emulsifiers is particularly preferred. Suitable other emulsifiers are, for example, nonionic surfactants from at least one of the following groups:
products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;
alkyl and/or alkenyl oligoglycosides containing 8 to 22 carbon atoms in the alkyl group and ethoxylated analogs thereof;
addition products of 1 to 15 mol ethylene oxide onto castor oil and/or hydrogenated castor oil;
addition products of 15 to 60 mol ethylene oxide onto castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;
partial esters of polyglycerol (average degree of self-condensation 2 to 8), polyethylene glycol (molecular weight 400 to 5000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol;
mono-, di- and trialkyl phosphates and mono-, di- and/or tri-PEG-alkyl phosphates and salts thereof;
wool wax alcohols;
polysiloxane/polyalkyl/polyether copolymers and corresponding derivatives;
block copolymers, for example Polyethyleneglycol-30 Dipolyhydroxystearate;
polymer emulsifiers, for example Pemulen types (TR-1, TR-2) of Goodrich;
polyalkylene glycols and
glycerol carbonate.

The sterol and/or stanol esters of ethercarboxylic acids may also be used as a cosmetic active component.

In addition, the sterol and/or stanol esters of ethercarboxylic acids claimed in claim 1 may be used as a hypocholesterolaemic agent, particularly in foods and/or food supplements. For example, the compounds may be used in foods, such as margarine, salad dressings and cooking oils according to the publications cited above, preferably in quantities of 0.1 to 10% by weight, based on the particular product. The cholesterol level in the blood of humans and animals is reduced by the addition of the esters mentioned, the cleavage of the esters by the lipases in the body and the resulting release of the sterol.

### EXAMPLES

Generol R™, marketed by Cognis Deutschland GmbH & Co. KG, is a mixture of phytosterols based on 40 to 60% by weight β-sitosterol, 30 to 45% by weight campesterol and 8 to 18% by weight brassicasterol, 0 to 5% by weight stigmasterol, 0 to 5% by weight stigmastanol, 0 to 5% by weight ergostanol and a total content of 90 to 100% by weight. Melting range 130-145° C.

### Example 1

In a reaction flask, 0.5 mol (212.3 g) Generol™ of Cognis Deutschland GmbH & Co. KG (characteristics: OHV 132; average molecular weight calculated from the OHV=425.1 g/mol) and 0.65 mol (308.1 g) of the ethercarboxylic acid Akypo™ LF 1 of Kao (n-octanol-based ethercarboxylic acid ethoxylated with 5 mol ethylene oxide; acid value 118 and average molecular weight 474.4 g/mol) were dried for 1 hour at 140° C. 0.8 g (0.15% by weight) calcium oxide were then added as esterification catalyst and reacted in a water jet vacuum for 5 hours at 200° C. After cooling to ca. 60° C., the product was filtered off through a nutsch filter. An ethercarboxylic acid liquid at room temperature with the characteristic data set out in Table 1 was obtained. The difference between the saponification and acid values clearly shows that esterification took place.

The residual acid value is explained by the incomplete reaction.

### Example 2

0.5 mol Generol R™ of Cognis Deutschland GmbH & Co. KG and 0.65 mol Akypo LF 2™ (octanol-based ethercarboxylic acid ethoxylated with 8 mol ethylene oxide; acid value 96.4) were reacted as in Example 1. A wax-like ethercarboxylic acid ester with a melting range of 30 to 40° C. and the characteristic data shown in Table 1 was obtained.

### Example 3

0.5 mol Generol R™ of Cognis Deutschland GmbH & Co. KG and 0.65 mol Akypo RLM 100™ (C_{12/14}-alcohol-based ethercarboxylic acid ethoxylated with 10 mol ethylene oxide; active substance content 85% by weight; acid value 74) were reacted as in Example 1. A wax-like ethercarboxylic acid ester with a melting range of 30 to 40° C. and the characteristic data shown in Table 1 was obtained.

### Example 4

90 g water were added to 10 g of the ethercarboxylic acid esters prepared in Examples 1 to 3 and stirred. Aqueous beige-colored emulsions with little sediment were obtained in every case. Since no other emulsifiers had to be added, the esters are self-emulsifying in water.

The characteristic data of the resulting ethercarboxylic acid esters of the sterol mixture used are set out in Table 1 below.

The saponification value (SV) was determined to DGF C-V 3.

The hydroxyl value (OHV) was determined to DGF C-V 17a.

The acid value (AV) was determined to DIN 53402.

TABLE 1
Characteristic data

| Example | Raw material | SV | OHV | AV |
|---|---|---|---|---|
| 1 | Akypo ™ LF1 | 76 | 12.8 | 5.2 |
| 2 | Akypo LF 2 ™ | 66 | 11.1 | 9.1 |
| 3 | Akypo RLM 100 ™ | 60 | 8.3 | 5.9 |

### Example 5

Preparation of O/W Emulsions

O/W emulsions were prepared by a standard hot process by heating the oil phase containing emulsifiers, fatty alcohols and an emollient to 80° C. and homogenizing with a stirrer. A glycerol/water phase similarly heated to 80° C was then introduced with stirring at that temperature. The mixture was cooled while stirring, formalin being added as a preservative.

The emulsions were tested for viscosity (Brookfield RVF, spindle 5, 10 r.p.m.) and stability (optical scale of 1 to 5, 1 being the best score; after storage for 1 week at −5 and 40° C.).

The compositions of the o/w emulsions (quantities in g) and their evaluation are shown in Table 2 below.

TABLE 2
O/W Emulsions

| | | | Example for |
|---|---|---|---|
| | Example | Example | comparison |
| | 5a (in g) | 5b | with 5a |
| Oil phase: | | | |
| Sterol + 10EO | 0.00 | 0.00 | 2.00 |
| C12/14 carboxylic acid ester | 2.00 | 1.00 | 0.00 |
| of sterol + 10EO, Example 3 | 0.00 | 1.00 | 0.00 |
| Co-emulsifier (C16/18 fatty | 5.00 | 5.00 | 5.00 |
| alcohol + 2EO) | | | |
| C16/18 fatty alcohol | 16.00 | 16.00 | 16.00 |
| Emollient (lauryl caprylate) | | | |
| Glycerol (purity 99%) | 3.00 | 3.00 | 3.00 |
| Water, dist. | to 100 | to 100 | to 100 |
| Formalin (37%) | 0.15 | 0.15 | 0.15 |
| Viscosity | | | Phase separation |
| After 1 day | 6000 | 4400 | after preparation, |
| After 1 week | 8400 | 7200 | no viscosity |
| | | | measurable |
| Stability (scale 1 to 5) | | | Phase separation |
| After 1 week at −5° C. | 1 | 1 | after preparation, |
| After 1 week at 40° C. | 1 | 1 | no stability |

It can be seen from Table 2 that the ethercarboxylic acid esters of the sterols according to the invention have emulsifier properties on their own (Example 5a) and in combination with co-emulsifiers (Example 5b).

### Example 6

Biotransformation of the Sterol-EO Ester of Example 1 to Steroid Precursors

Yeast extract and potato dextrose agar (Difco, USA), glycerol with p.a. purity (Riedel de Haen, Germany), Tween 20 (polyethylene glycol sorbitan monolaurate) and 4-androsten-3,17-dione (AD) and 1,4-androstadien-3,17-dione (ADD) (Sigma, USA) were used.

Cell culture: Mycobacterium sp. NRRL B-3805 cells were kept in potato dextrose agar in a slant culture tube (42 gl⁻¹). Inoculums: Mycobacterium sp. NRRL B-3805 cells were cultivated for three days at 30° C. in a complex medium A of yeast extract (10 gl⁻¹), glycerol (10 gl⁻¹), sterol ester (1.0 gl⁻¹ unless otherwise indicated), Tween 20 (0.8 gl⁻¹) in a potassium phosphate buffer solution with a pH of 7 (20 mom).

The cells were then introduced into 20 ml or 160 ml of the same medium A. After growth for 24 hours, medium A was used to inoculate 1.4 l (2 l fermenter vessel (Braun Bio stat MD). The fermentation process was accompanied by aeration (aeration rate 0.5 vim) and the pH of the fermentation medium was adjusted to pH 7 by addition of NaOH (2 N) or H₂SO₄ (2 N). Samples were taken to monitor the AD concentration formed.

Analysis Methods

Samples of the fermentation medium were routinely taken at periodic intervals and extracted with twice the volume of a solution of progesterone (0.2 gl⁻¹) in n-heptane as internal standard. The organic phase was analyzed for its sterol/steroid content by HPLC.

A sample of the ester of Example 1 was saponified as follows: ca. 1 g of each ester was added to 25 ml of a 0.5 M ethanolic solution of KOH and incubated overnight at 28° C. 25 ml of an NaCl (5% by weight) solution was then added and the whole was extracted with 25 ml diethyl ether. The organic phase was concentrated by evaporation and a dry residue was obtained. Ca. 6 mg of the saponified sample were dissolved in 2 ml of the internal standard solution and quantitatively analyzed for sterols by HPLC.

During the fermentation test, samples were taken and saponified as follows: 2.5 ml of a 0.5 M ethanolic KOH solution were added to 500 ml of a fermentation sample and incubated overnight at 28° C. 25 ml of a 5% by weight NaCl solution were then added and the whole was extracted with 25 ml diethyl ether. The organic phase was concentrated by evaporation, the dry residue obtained was dissolved in 1 ml internal standard solution and quantitatively analyzed for sterols by HPLC.

The HPLC analysis (Lichrospher Si-60 column, 10 μm particle size) was carried out with a flow rate of 1 ml min⁻¹ in order to determine the substrate and product concentration (UV detection at 215 and 254 nm). The mobile phase consisted of (v/v) n-heptane (96):ethanol (4).

After a fermentation time of 130 hours, ca. 0.6 mM 4-androsten-3,17-dione had been produced. This result shows that Mycobacterium sp. NRRL B-3805 cells are capable of converting the esters into AD in accordance with the invention.

1-11. (canceled)

12. Sterol and/or stanol esters of ethercarboxylic acids corresponding to general formula (I):
R(OAlk)ₙOCH₂COOH (I)
in which R is a C₁₋₅₀ alkyl, alkenyl or alkylphenyl group derived from an alcohol, OAlk stands for a ring-opened ethylene oxide, propylene oxide or butylene oxide unit, and n is a number from 0 to 100.

13. Sterol and/or stanol esters according to claim 12, wherein R is a linear C₁₋₃₆ alkyl group, optionally containing other functional groups derived from an alcohol.

14. Sterol and/or stanol esters according to claim 12, wherein R is a linear C₁₋₁₈ alkyl group derived from a monofunctional alcohol.

15. Sterol and/or stanol esters according to claim 12, wherein R is a linear hydroxyl-terminated C₂₋₃₆ alkyl group derived from a difunctional alcohol.

16. Sterol and/or stanol esters according to claim 12, wherein bAlk stands for the ring-opened ethylene oxide unit OCH₂CH₂.

17. Sterol and/or stanol esters according to claim 12, wherein n is a number from 1 to 20.

18. Sterol and/or stanol esters according to claim 12, wherein the sterol or stanol is a phytosterol or phytostanol.

19. Sterol and/or stanol esters according to claim 18, wherein the phytosterol or phytostanol comprises one or more compounds selected from β-sitosterol, campesterol, stigmasterol, brassicasterol, stigmasterol and campestanol.

20. Sterol and/or stanol esters according to claim 18, wherein the phytosterol or phytostanol is derived from rapeseed oil.

21. A process for the production of sterol and/or stanol esters of ethercarboxylic acids, comprising esterifying sterols, stanols or mixtures thereof with ethercarboxylic acids or their salts corresponding to formula (II):
R(OAlk)ₙOCH₂COOX (II)
in which R is a C₁₋₅₀ alkyl, alkenyl, or alkylphenyl group derived from an alcohol, OAlk stands for a ring-opened ethylene oxide, propylene oxide or butylene oxide unit, n is a number from 0 to 100 and X is hydrogen or an alkali metal, in the presence of an esterification catalyst.

22. The process according to claim 21, wherein R is a linear C₁₋₃₆ alkyl group, optionally containing other functional groups derived from an alcohol.

23. The process according to claim 21, wherein OAlk stands for the ring-opened ethylene oxide unit OCH₂CH₂.

24. The process according to claim 21, wherein n is a number from 1 to 20.

25. The process according to claim 21, wherein the sterol or stanol is a phytosterol or phytostanol.

26. The process according to claim 25, wherein the phytosterol or phytostanol is derived from rapeseed oil.

27. A cosmetic composition comprising a sterol and/or stanol ester of ether carboxylic acids according to claim 12.

28. A food or food supplement comprising a sterol and/or stanol ester of ether carboxylic acids according to claim 12.

29. A food or food supplement according to claim 28, wherein the sterol or stanol is a phytosterol or phytostanol.

30. A food or food supplement according to claim 29, wherein the phytosterol or phytostanol is derived from rapeseed oil.

31. An emulsifier or co-emulsifier comprising a sterol and/or stanol ester of ethercarboxylic acids corresponding to general formula (I):
R(OAlk)ₙOCH₂COOH (I)
in which R is a C₁₋₅₀ alkyl, alkenyl or alkylphenyl group derived from an alcohol, OAlk stands for a ring-opened ethylene oxide, propylene oxide or butylene oxide unit, and n is a number from 0 to 100.

32. An emulsifier or co-emulsifier according to claim 21, wherein R is a linear C₁₋₃₆ alkyl group, optionally containing other functional groups derived from an alcohol.

33. An emulsifier or co-emulsifier according to claim 21, wherein OAlk stands for the ring-opened ethylene oxide unit OCH₂CH₂.

34. An emulsifier or co-emulsifier according to claim 21, wherein n is a number from 1 to 20.

35. An emulsifier or co-emulsifier according to claim 21, wherein the sterol or stanol is a phytosterol or phytostanol.

36. An emulsifier or co-emulsifier according to claim 21, wherein the phytosterol or phytostanol is derived from rapeseed oil.
